(19) United States
(12) Patent Application Publication
(10) Pub. No.: US 2022/0398465 A1
(43) Pub. Date: Dec. 15, 2022

(54) METHOD AND APPARATUS FOR ESTABLISHING RISK PREDICTION MODEL AS WELL AS REGIONAL RISK PREDICTION METHOD AND APPARATUS

(71) Applicant: BEIJING BAIDU NETCOM SCIENCE AND TECHNOLOGY CO., LTD., Beijing (CN)

(72) Inventors: Jizhou HUANG, Beijing (CN); Jingbo ZHOU, Beijing (CN); An ZHUO, Beijing (CN); Ji LIU, Beijing (CN); Haoyi XIONG, Beijing (CN); Dejing DOU, Beijing (CN); Haifeng WANG, Beijing (CN)

(73) Assignee: BEIJING BAIDU NETCOM SCIENCE AND TECHNOLOGY CO., LTD., Beijing (CN)

(21) Appl. No.: 17/620,820

(22) PCT Filed: Jun. 2, 2021
(86) PCT No.: PCT/CN2021/097958

§ 371 Date: Dec. 20, 2021

(30) Foreign Application Priority Data
Dec. 21, 2020 (CN) 202011515953.3

(51) Int. Cl. G06N 5/02

(52) U.S. Cl. CPC G06N 5/022

(57) ABSTRACT

A technical solution relates to a big data technology in the field of artificial intelligence technologies. The technical solution includes: acquiring training data including annotation results of a risk grade of each sample region and a risk grade of a district to which each sample region belongs; and training an initial model including an encoder, a discriminator and a classifier using the training data, and obtaining the risk prediction model using the encoder and the classifier after the training process. The encoder performs a coding operation using region features of the sample regions to obtains a feature representation of each sample region; the discriminator identifies the risk grade of the district to which the sample region belongs according to the feature representation of the sample region; the classifier identifies the risk grade of the sample region according to the feature representation of the sample region.

This application is the national phase of PCT Application No. PCT/CN2021/097958 filed on Jun. 2, 2021, which claims priority to Chinese Patent Application No. 202011515953.3, filed on Dec. 21, 2020, entitled “Method and Apparatus for Establishing Risk Prediction Model As Well As Regional Risk Prediction Method and Apparatus”, which are hereby incorporated in their entireties by reference herein.

### TECHNICAL FIELD

The present disclosure relates to the field of computer application technologies, and particularly to a big data technology in the field of artificial intelligence technologies.

### BACKGROUND

A public emergency, such as epidemic spread, a biological disaster, a meteorological disaster, has a great influence on production, living and even safety of people. If a regional risk could be predicted timely and accurately, an emergency hazard might be effectively prevented from being spread, and targeted preventive measures may be taken, thus having a great significance.

### SUMMARY

According to an embodiment of the present disclosure, there is provided a method for establishing a risk prediction model, including:

acquiring training data, the training data including a sample region set and annotation results of a risk grade of each sample region in the sample region set and a risk grade of a district to which each sample region belongs; and

training an initial model including an encoder, a discriminator and a classifier using the training data, and obtaining the risk prediction model using the encoder and the classifier in the initial model after the training process;

where the encoder performs a coding operation using region features of the sample regions to obtain a feature representation of each sample region; the discriminator identifies the risk grade of the district to which the sample region belongs according to the feature representation of the sample region; the classifier identifies the risk grade of the sample region according to the feature representation of the sample region; the initial model has training targets of minimizing a difference of identification of the sample regions belonging to the districts with different risk grades by the discriminator, and minimizing a difference between the identification result of the sample region by the classifier and the annotation result.

According to an embodiment of the present disclosure, there is provided a regional risk prediction method, including:

extracting region features of a target block; and

inputting the region features into a risk prediction model, and determining a risk grade of the target region according to a result output by the risk prediction model;

where the risk prediction model is pre-established using the method as described above.

According to some embodiments of the present disclosure, there is provided an electronic device, including:

at least one processor; and

a memory connected with the at least one processor communicatively;

where the memory stores instructions executable by the at least one processor to enable the at least one processor to perform the method as mentioned above.

According to some embodiments of the present disclosure, there is provided a non-transitory computer readable storage medium including computer instructions, which, when executed by a computer, cause the computer to perform the method as mentioned above.

It should be understood that the statements in this section are not intended to identify key or critical features of the embodiments of the present disclosure, nor limit the scope of the present disclosure. Other features of the present disclosure will become apparent from the following description.

### BRIEF DESCRIPTION OF THE DRAWINGS

The drawings are used for better understanding the present solution and do not constitute a limitation of the present disclosure. In the drawings,

FIG. 1 is a flow chart of a method for establishing a risk prediction model according to an embodiment of the present disclosure;

FIG. 2 is a schematic structural diagram of a trained initial model according to an embodiment of the present disclosure;

FIG. 3 is a schematic structural diagram of the risk prediction model according to an embodiment of the present disclosure;

FIG. 4 is a regional risk prediction method according to an embodiment of the present disclosure;

FIG. 5 is a structural diagram of an apparatus for establishing a risk prediction model according to the present disclosure;

FIG. 6 is a structural diagram of a regional risk prediction apparatus according to the present disclosure; and

FIG. 7 is a block diagram of an electronic device configured to implement the embodiment of the present disclosure.

### DETAILED DESCRIPTION OF EMBODIMENTS

The following part will illustrate exemplary embodiments of the present disclosure with reference to the drawings, including various details of the embodiments of the present disclosure for a better understanding. The embodiments should be regarded only as exemplary ones. Therefore, those skilled in the art should appreciate that various changes or modifications can be made with respect to the embodiments described herein without departing from the scope and spirit of the present disclosure. Similarly, for clarity and conciseness, the descriptions of the known functions and structures are omitted in the descriptions below.

In an existing method for predicting a risk of a public emergency, such as an epidemic, a prediction is performed mainly by an infectious disease model using for example, a temporal and spatial distribution of infected users, a transmission speed of an infectious disease, a transmission path, or the like. However, such a model requires sufficient understanding and an accurate grasp of the epidemic as well as a sufficient professional knowledge background. However, spread of the epidemic is often sudden, and the onset of a disease is delayed (for example, there exists an incubation period, and a patient has no typical symptom in the incubation period), such that a risk prediction may have insufficient accuracy. In addition, such an infectious disease model is usually able to perform a prediction for a district where epidemic spread has occurred, but unable to perform a prediction for a district where the epidemic has not occurred.

In a method for establishing a risk prediction model according to the present disclosure, by learning features of regions in districts with different risk grades and features of the regions with different risk grades, a risk grade of a region with unknown risk conditions may be predicted based on the features. The method according to the present disclosure will be described below in detail in conjunction with an embodiment.

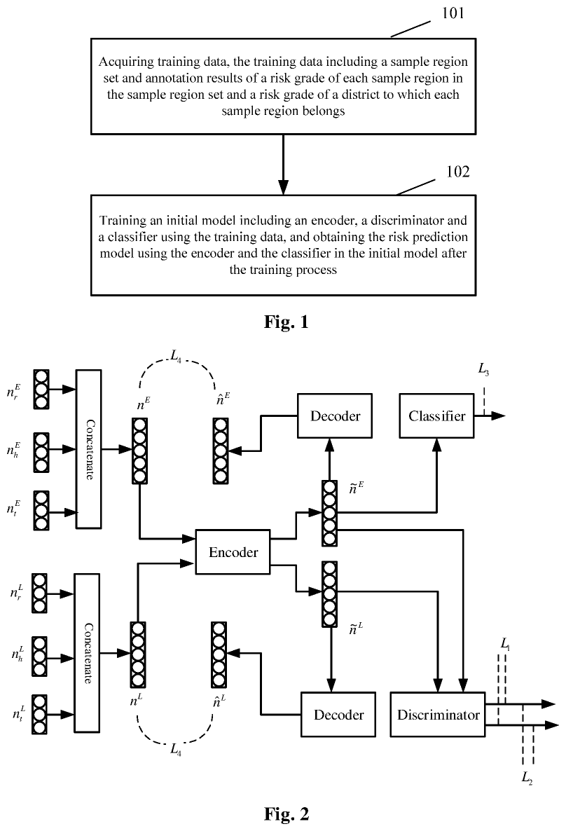

FIG. 1 is a flow chart of a method for establishing a risk prediction model according to an embodiment of the present disclosure, and as shown in FIG. 1, the method may include the following steps:

101: acquiring training data, the training data including a sample region set and annotation results of a risk grade of each sample region in the sample region set and a risk grade of a district to which each sample region belongs.

Regions with various risk grades in districts with various risk grades may be collected in advance as samples in the present disclosure. The district has a greater range than the region. For example, the district may be a province, a city, an administrative district, or the like. The region may be a block, a street, a school, a building, a factory, or the like.

The risk grade of the district may be divided into two types, such as a high risk grade and a low risk grade, and may also be divided into a plurality of types, such as a high risk grade, a medium risk grade, a low risk grade, a risk-free grade, or the like. The risk grade of the region may also be divided into two types, such as a high risk grade and a low risk grade, and may also be divided into a plurality of types, such as a high risk grade, a medium risk grade, a low risk grade, a risk-free grade, or the like. A specific division manner and specific division granularity are not limited in the present disclosure.

The risk grade of the district of each sample region and the risk grade of each sample region may be labeled in advance in the training data to be used in a subsequent model training process.

102: training an initial model including an encoder, a discriminator and a classifier using the training data, and obtaining the risk prediction model using the encoder and the classifier in the initial model after the training process.

The encoder performs a coding operation using region features extracted from the sample regions to obtain a feature representation of each sample region; the discriminator identifies the risk grade of the district to which the sample region belongs according to the feature representation of the sample region; the classifier identifies the risk grade of the sample region according to the feature representation of the sample region; the initial model has training targets of minimizing a difference of identification of the sample regions belonging to the districts with different risk grades by the discriminator, and minimizing a difference between the identification result of the sample region by the classifier and the annotation result.

From the above technical solution, the present disclosure provides the method for establishing a risk prediction model, and a risk prediction of the target region may be realized based on the established risk prediction model, thereby effectively preventing spread of an emergency hazard, and taking targeted preventive measures.

The steps in the above-mentioned embodiment are described in detail below with reference to an embodiment. In addition, since the method according to the present disclosure may be well applied to the epidemic risk prediction, the following embodiment will be described with the epidemic risk prediction as an example.

In the above-mentioned step 101, assuming that cities are divided into high risk cities and low risk cities in advance, some high risk blocks and some low risk blocks are selected from the known high risk cities and some low risk blocks are selected from the known low risk cities (usually, there are no high risk blocks in the low risk cities). A specific division manner is determined based on infection and spread conditions of the epidemic in the cities and the blocks. The sample region set is formed by the selected blocks, and the risk grades of the cities and the risk grades of the blocks are labeled for the blocks respectively, so as to constitute the training data.

Still further, the region features may be extracted separately for each block in the training data. The region feature extracted in the present disclosure may include at least one of a surrounding preset-type POI feature, a demographic feature, and a user travel feature. Unlike the existing infectious disease model, these region features employed in the present disclosure are not relevant to confirmed cases, and therefore, the prediction of a block risk may be performed in epidemic non-outbreak cities without prior experiences. The several features are described in detail below.

Surrounding preset-type POI feature:

living facilities around a block are usually related to a probability that the block is affected by an epidemic. For example, a block may be at a high risk due to a lack of basic living facilities, as residents may go farther to obtain living needs, and then, there exists a road infection possibility. Usually, the block lacking the basic living facilities lacks good management, also resulting in a high infection risk.

Based on the above considerations, the features of a preset type of POIs around a block may include, but are not limited to, the following two types:

The first type: information of a distance between the block and the nearest POI of the preset type. More than one type of POIs may be preset in the present disclosure, such as hospitals, clinics, schools, preschool educational institutions, bus stations, subway stations, airports, train stations, long-distance passenger stations, shopping malls, supermarkets, markets, shops, police offices, scenic spots, or the like. The features may be characterized by distances of the block from the nearest hospital, the nearest clinic, the nearest school, or the like.

The second type: a completeness degree of the living facilities in a preset distance range of the block. The completeness degree of the living facilities within, for example, 1 km may be adopted as one of the features in the present disclosure. That is, an evaluation may be performed based on conditions of hospitals, bus stations, supermarkets, shopping malls, markets, or the like, within 1 km. For example, 1 indicates a highest completeness degree, and 0 indicates a lowest completeness degree.

Demographic Feature:

Since the epidemic is usually spread from person to person, the risk is required to be predicted in consideration of population density. Usually, the block with higher population density has a higher infection risk than the block with lower population density. Therefore, the population density may be taken as one of the demographic features.

In addition, different commuting distances also have a certain influence on the risk of the epidemic, and therefore, a distribution of the commuting distances of the block may be taken as one of the demographic features. As one implementation, an average commuting distance of the block may be used for characterization. The commuting distance may refer to a distance from a work place, a distance from a school, or the like.

Usually, different populations have different infection probabilities when exposed to the epidemic; for example, older and younger people are often more susceptible to infection due to weak immune systems. As another example, highly educated people have a higher degree of risk awareness and prevention, and therefore have a relatively lower infection probability. Based on this consideration, at least one of an age distribution, a gender distribution, an income distribution, a consumption ability distribution, an education level distribution, a marital status distribution, a life stage distribution, a job type distribution, an industry type distribution, or the like, may be selected as the demographic feature.

User Travel Feature:

Some related researches prove that user travel behaviors are usually closely related to epidemic spread. The user travel features involved in the present disclosure may include, but are not limited to, at least one of the following types:

The first type: a travel mode. For example, travel modes, such as walking, riding, public traffic, a private car, or the like, may be predefined.

The second type: a starting point-destination mode distribution. Information, such as a type of a destination, a distance between a starting point and the destination, or the like, may be included. The destinations may be classified into hospitals, restaurants, hotels, schools, or the like, in advance, a plurality of distance buckets are defined in advance, for example, 0 km-3 km, 3 km-10 km, 10 km-20 km, or the like, and the distance between the starting point and the destination is mapped to the corresponding distance bucket, which is taken as the feature.

The third type: a starting point-travel mode-destination mode distribution. The starting point refers to the current block, the travel mode and the destination type may be predefined, and then, top N combinations of counted combinations formed by the travel modes and the destination types of the block are used as the features. N is a preset positive integer, for example, 20.

It is observed that the above-mentioned features are relatively easy to obtain under any condition, and social and economic conditions as well as characteristics of spatial interaction activities of one region may be reflected at fine granularity of blocks, thereby realizing high-risk region identification at the fine granularity, and reducing a social cost.

The above-mentioned step 102 will be described below in detail in conjunction with an embodiment. First, a structure of the initial model used in the training process is described. As shown in FIG. 2, the initial model may include an encoder, a discriminator and a classifier, and may further include a decoder.

The region features extracted from the sample blocks are used as input of the encoder, and since the sample blocks belonging to the cities with different risk grades may be used in an actual training process, the sample blocks of the high risk cities and the sample blocks of the low risk cities are taken as examples in this embodiment. The surrounding preset-type POI feature, the demographic feature and the user travel feature of the sample block of the high risk city are represented by nᵣ^{E}, nₕ^{E} and nₜ^{E} respectively, and the surrounding preset-type POI feature, the demographic feature and the user travel feature of the sample block of the low risk city are represented by n^{L}, nₕ^{L} and nₜ^{L} respectively. nᵣ^{E}, nₕ^{E} and nₜ^{E} are fused, for example, are concatenated, to obtain the feature n^{E} of the sample block of the high risk city. nᵣ^{L}, nₕ^{L} and nₜ^{L} are fused, for example, are concatenated to obtain the feature n^{L} of the sample block of the low risk city.

n^{E} is used as the input of the encoder and encoded by the encoder to obtain the feature representation ñe of the sample block of the high risk city. similarly, n^{L} is used as the input of the encoder and encoded by the encoder to obtain the feature representation ñ^{L} of the sample block of the low risk city. The encoder may be regarded to perform transformation on an input feature vector to obtain a new probability distribution.

In general, if experiences are wished to be learned from cities having massive outbreaks (i.e., high risk cities), these experiences are often required to have some commonality between different cities, and are not unique characteristics of the cities. How to learn these common features is a very important problem in the model training process. In the present disclosure, this problem is solved by training a discrimination model.

The discrimination model has functions of discriminating the risk grade of the city from which the feature representation originates according to the input n E, and discriminating the risk grade of the city from which the feature representation originates according to the input ñ^{L}. The training process has an important training target of, after the coding operation of the encoder, enabling the obtained feature representation to make the discrimination model unable to distinguish the city from which the feature representation originates as far as possible, that is, minimizing a difference of identification by the discriminator of the sample regions belonging to the districts with different risk grades, which enables the encoder to learn the common features between the cities. From the training target, a loss function, referred to as a second loss function L₂, may be constructed, such as:

L₂=−[log(D(ñ^{E}))+log(D(ñ^{L}))

where D( ) represent an identification result of the discrimination model.

Further, besides learning the common features between the cities, the discrimination model is required to guarantee its own function (i.e., identification of the risk grade of the city from which the feature representation originates). Therefore, a loss function, referred to as a first loss function L₁, may be constructed in an adversarial learning manner, and the loss function is used for training the discrimination model to minimize the difference between the result of identification of the sample region by the discriminator and the annotation result. This loss function may be, for example,

L₁=−[log(D(ñ^{E}))+log(1−D(ñ^{L}))

In the adversarial learning process, the discriminator continuously learns how to distinguish the risk grades of the cities from which ñ^{E} and ñ^{L} originate under an influence of L₁, which may result in an increase of L₂. Then, the encoder learns the common features as far as possible under the influence of L₂, so as to reduce L₂, such that the encoder and the discriminator perform continuous adversarial behaviors in the learning process, so as to finally reach a balance. At this point, the discriminator is unable to distinguish the sample blocks in the high risk cities and the low risk cities, and the encoder learns the common features between the sample blocks in the high risk cities and the sample blocks in the low risk cities.

Using the above-mentioned learning method, the common features between the sample blocks of the high risk cities and the sample blocks of the low risk cities may be learned, but the features of the sample blocks are not able to be learned to guide the identification of the risk grades of the blocks. Therefore, in the initial model, the risk grade of the block is identified by the classifier.

The classifier identifies the risk grade of the corresponding sample block according to ñ^{E}, with a training target of minimizing the difference between the result of the identification of the sample region by the classifier and the annotation result. In this regard, a loss function, i.e., a third loss function, L₃ may be constructed. This loss function may be, for example,

L₃=−y^{E} log(C(ñ^{E}))−(1−y^{E})log(1−C(ñ^{E}))

where y^{E} represents the annotation result, and C(ñ^{E}) represents the result of the identification of ñ^{E} by the classifier.

The encoder and the classifier are optimized using the loss function, such that the encoder further learns the features capable of guiding the identification of the risk grade of the block on the basis of learning the common features between the cities. The classifier is guided to learn a capability of identifying the risk grade of the block. It should be additionally noted that the above-mentioned classifier is described with binary classification as an example, but a multi-classification classifier may be used in an actual model.

Still further, in order to enable the encoder to learn the features of the block as far as possible, an encoder-decoder framework is added in the present disclosure for feature reconstruction.

The encoder has a function of reconstructing the features of the region using the input feature representation of the sample block. That is, n^{E} is reconstructed to obtain the vector representation \hat{n}^{E} with a consistent dimension with n^{E}. n^{L} is reconstructed to obtain the vector representation \hat{n}^{L} with a consistent dimension with n^{L}. The encoder has an optimal target of recovering the original vector representation, that is, minimizing the difference between the reconstructed region features and the region features extracted from the sample region. Accordingly, a fourth loss function L₄ may be constructed. This loss function may be, for example,

L₄=∥\hat{n}^{E},n^{E}∥₂+∥\hat{n}^{L},n^{L}∥₂L

The encoder and the decoder are optimized using L₄, such that the feature representation learned by the encoder still has the capability of describing the characteristics of one block.

In conclusion, it is observed that, as an embodiment, in the process of training the initial model, the above-mentioned four loss functions are used to optimize and update the model parameters. Specifically, in each iteration process, parameters of the discriminator are optimized and updated using L₁, parameters of the encoder are optimized and updated using L₂, L₃ and L₄, and parameters of the classifier and the decoder are optimized and updated using L₃, and L₄.

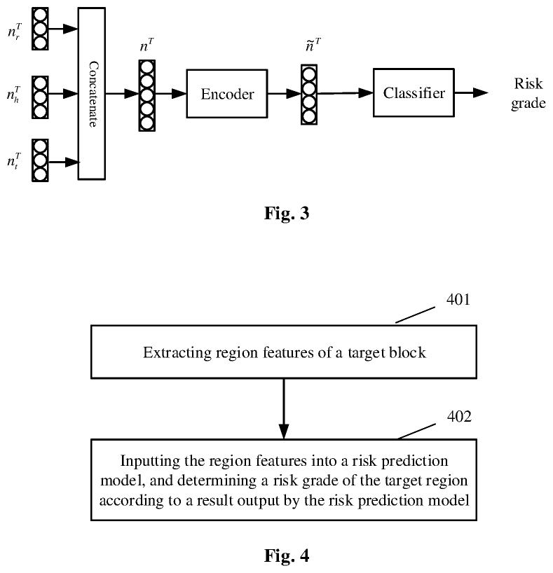

After the initial model is trained, for example, after the model converges or a preset iteration number is reached, the risk prediction model is obtained by the trained encoder and the trained classifier. That is, although the discriminator and the decoder are used in the training process to assist the training operation, only the encoder and the classifier are used in the actually obtained risk prediction model, which is shown in FIG. 3.

FIG. 4 is a regional risk prediction method according to an embodiment of the present disclosure, and the method is implemented based on the above-mentioned established risk prediction model. As shown in FIG. 4, the method includes:

401: extracting region features of a target block.

A manner of extracting the region features in the step is consistent with that of the region features adopted in the process of training the risk prediction model. The region feature may also include at least one of a surrounding preset-type POI feature, a demographic feature, and a user travel feature. For specific content of the region feature, reference is made to the related description in the embodiment shown in FIG. 1, which is not repeated herein.

402: inputting the region features into a risk prediction model, and determining a risk grade of the target region according to a result output by the risk prediction model.

As shown in FIG. 3, the surrounding preset-type POI feature, the demographic feature, and the user travel feature of the sample block of the high risk city are represented by nᵣ^{T}, nₕ^{T} and nₜ^{T} respectively. nᵣ^{T}, nₕ^{T} and nₜ^{T} are fused, for example, are concatenated to obtain the feature n of the target block.

n^{T} is used as the input of the encoder and encoded by the encoder to obtain the feature representation ñ^{T} of the target block. The classifier identifies the risk grade of the corresponding sample block according to ñ^{T}.

It is observed that, in the above-mentioned process of predicting the risk grade of the target region, information of the district to which the target region belongs is not required, and the prediction of the risk grade is independent of the district.

As a typical application scenario, the present disclosure may be used to predict the risk grade of the region during epidemic spread. With the solution, potential high risk regions may be identified in districts without massive epidemic outbreaks, thereby having a great guiding significance for prevention and control of the epidemic.

The method according to the present disclosure is described above in detail, and an apparatus according to the present disclosure will be described below in detail in conjunction with an embodiment.

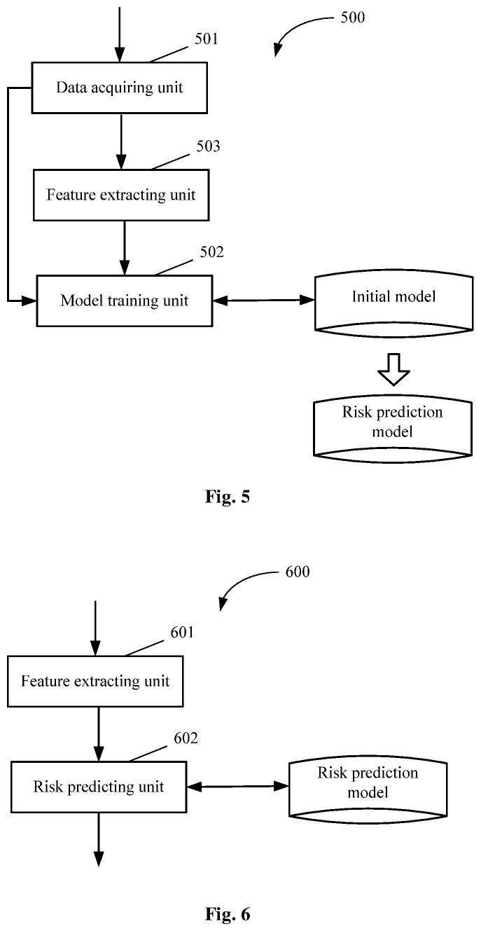

FIG. 5 is a structural diagram of an apparatus for establishing a risk prediction model according to the present disclosure; the apparatus may be configured as an application located at a server, or a functional unit, such as a plug-in or software development kit (SDK) located in the application of the server, or the like, or be located at a computer terminal with high computing power, which is not particularly limited in the embodiment of the present disclosure. As shown in FIG. 5, the apparatus 500 may include a data acquiring unit 501 and a model training unit 502, and may further include a feature extracting unit 503. The main functions of each constitutional unit are as follows.

The data acquiring unit 501 is configured to acquire training data, the training data including a sample region set and annotation results of a risk grade of each sample region in the sample region set and a risk grade of a district to which each sample region belongs.

The model training unit 502 is configured to train an initial model including an encoder, a discriminator and a classifier using the training data, and obtain the risk prediction model using the encoder and the classifier in the initial model after the training process.

The encoder performs a coding operation using region features of the sample regions to obtain a feature representation of each sample region; the discriminator identifies the risk grade of the district to which the sample region belongs according to the feature representation of the sample region; the classifier identifies the risk grade of the sample region according to the feature representation of the sample region; the initial model has training targets of minimizing a difference of identification of the sample regions belonging to the districts with different risk grades by the discriminator, and minimizing a difference between the identification result of the sample region by the classifier and the annotation result.

The feature extracting unit 503 is configured to acquire the region feature of the sample region, including at least one of: a surrounding preset-type POI feature, a demographic feature, and a user travel feature.

The surrounding preset-type POI feature includes at least one of information of a distance between the sample region and a nearest POI of a preset type, and a completeness degree of living facilities in a preset distance range of the sample region.

The demographic feature includes at least one of a population density condition, a commuting distance distribution, an age distribution, a gender distribution, an income distribution, a consumption ability distribution, an education level distribution, a marital status distribution, a life stage distribution, a job type distribution and an industry type distribution.

The user travel feature includes at least one of a travel mode, a starting point-destination mode distribution, and a starting point-travel mode-destination mode distribution.

As an embodiment, the above-mentioned initial model may further include a decoder. The decoder reconstructs the region feature according to the feature representation of the sample region; the training process also has a target of minimizing a difference between the region feature reconstructed by the decoder and the region feature extracted from the sample region.

As an embodiment, in the process of training the initial model, the model training unit 502 optimizes parameters of the discriminator using a first loss function, optimizes parameters of the encoder using a second loss function, a third loss function and a fourth loss function, optimizes parameters of the classifier using the third loss function, and optimizes parameters of the decoder using the fourth loss function.

The first loss function is used to minimize a difference between a result of identification of the sample region by the discriminator and the annotation result.

The second loss function is used to minimize the difference of the identification of the sample regions belonging to the districts with different risk grades by the discriminator.

The third loss function is used to minimize the difference between the result of the identification of the sample region by the classifier and the annotation result.

The fourth loss function is used to minimize the difference between the region feature reconstructed by the decoder and the region feature extracted from the sample region.

FIG. 6 is a structural diagram of a regional risk prediction apparatus according to the present disclosure; the apparatus may be configured as an application located at a server, or a functional unit, such as a plug-in or software development kit (SDK) located in the application of the server, or the like, or be located at a computer terminal with high computing power, which is not particularly limited in the embodiment of the present disclosure. As shown in FIG. 6, the apparatus 600 may include a feature extracting unit 601 and a risk predicting unit 602. The main functions of each constitutional unit are as follows.

The feature extracting unit 601 is configured to extract region features of a target block.

The risk predicting unit 602 is configured to input the region features into a risk prediction model, and determine a risk grade of the target region according to a result output by the risk prediction model.

The risk prediction model is pre-established by the apparatus shown in FIG. 5.

As a typical application scenario, the risk grade of the region predicted by the above-mentioned regional risk prediction apparatus is a risk grade of epidemic spread.

The embodiments in the present disclosure are described progressively, and mutual reference may be made to same and similar parts among the embodiments, and each embodiment focuses on differences from other embodiments. In particular, since the apparatus embodiment is substantially similar to the method embodiment, the description is relatively simple, and reference may be made to the corresponding description of the method embodiment for relevant points.

It should be noted here that the present disclosure may be applied to a typical application scenario, such as the risk grade prediction of epidemic spread, but besides this application scenario, the present disclosure may also be reasonably expanded within the scope of the idea of the present disclosure to be applied to other scenarios. The correspondingly extracted region features may be different when the present disclosure is applied to other application scenarios.

According to the embodiment of the present disclosure, there are also provided an electronic device, a readable storage medium and a computer program product.

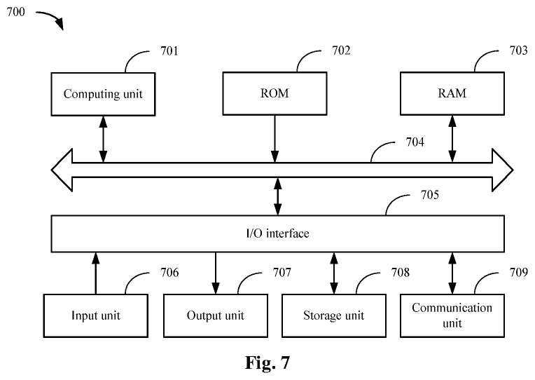

FIG. 7 is a block diagram of an electronic device configured to implement the embodiment of the present disclosure. The electronic device is intended to represent various forms of digital computers, such as laptop computers, desktop computers, workstations, personal digital assistants, servers, blade servers, mainframe computers, and other appropriate computers. The electronic device may also represent various forms of mobile apparatuses, such as personal digital processors, cellular telephones, smart phones, wearable devices, and other similar computing apparatuses. The components shown herein, their connections and relationships, and their functions, are meant to be exemplary only, and are not meant to limit implementation of the present disclosure described and/or claimed herein.

As shown in FIG. 7, the device 700 includes a computing unit 701 which may perform various appropriate actions and processing operations according to a computer program stored in a read only memory (ROM) 702 or a computer program loaded from a storage unit 708 into a random access memory (RAM) 703. Various programs and data necessary for the operation of the device 700 may be also stored in the RAM 703. The computing unit 701, the ROM 702, and the RAM 703 are connected with one other through a bus 704. An input/output (I/O) interface 705 is also connected to the bus 704.

The plural components in the device 700 are connected to the I/O interface 705, and include: an input unit 706, such as a keyboard, a mouse, or the like; an output unit 707, such as various types of displays, speakers, or the like; the storage unit 708, such as a magnetic disk, an optical disk, or the like; and a communication unit 709, such as a network card, a modem, a wireless communication transceiver, or the like. The communication unit 709 allows the device 700 to exchange information/data with other devices through a computer network, such as the Internet, and/or various telecommunication networks.

The computing unit 701 may be a variety of general and/or special purpose processing components with processing and computing capabilities. Some examples of the computing unit 701 include, but are not limited to, a central processing unit (CPU), a graphic processing unit (GPU), various dedicated artificial intelligence (AI) computing chips, various computing units running machine learning model algorithms, a digital signal processor (DSP), and any suitable processor, controller, microcontroller, or the like. The computing unit 701 performs the methods and processing operations described above, such as the method for establishing a risk prediction model or the regional risk prediction method. For example, in some embodiments, the method for establishing a risk prediction model or the regional risk prediction method may be implemented as a computer software program tangibly contained in a machine readable medium, such as the storage unit 708.

In some embodiments, part or all of the computer program may be loaded and/or installed into the device 700 via the ROM 502 and/or the communication unit 709. When the computer program is loaded into the RAM 703 and executed by the computing unit 701, one or more steps of the method for establishing a risk prediction model and the regional risk prediction method described above may be performed. Alternatively, in other embodiments, the computing unit 701 may be configured to perform the method for establishing a risk prediction model or the regional risk prediction method by any other suitable means (for example, by means of firmware).

Various implementations of the systems and technologies described herein may be implemented in digital electronic circuitry, integrated circuitry, field programmable gate arrays (FPGA), application specific integrated circuits (ASIC), application specific standard products (ASSP), systems on chips (SOC), complex programmable logic devices (CPLD), computer hardware, firmware, software, and/or combinations thereof.

The systems and technologies may be implemented in one or more computer programs which are executable and/or interpretable on a programmable system including at least one programmable processor, and the programmable processor may be special or general, and may receive data and instructions from, and transmit data and instructions to, a storage system, at least one input apparatus, and at least one output apparatus.

Program codes for implementing the method according to the present disclosure may be written in any combination of one or more programming languages. These program codes may be provided to a processor or a controller of a general purpose computer, a special purpose computer, or other programmable data processing apparatuses, such that the program code, when executed by the processor or the controller, causes functions/operations specified in the flowchart and/or the block diagram to be implemented. The program code may be executed entirely on a machine, partly on a machine, partly on a machine as a stand-alone software package and partly on a remote machine, or entirely on a remote machine or a server.

In the context of the present disclosure, the machine readable medium may be a tangible medium which may contain or store a program for use by or in connection with an instruction execution system, apparatus, or device. The machine readable medium may be a machine readable signal medium or a machine readable storage medium. The machine readable medium may include, but is not limited to, an electronic, magnetic, optical, electromagnetic, infrared, or semiconductor system, apparatus, or device, or any suitable combination of the foregoing. More specific examples of the machine readable storage medium may include an electrical connection based on one or more wires, a portable computer disk, a hard disk, a random access memory (RAM), a read only memory (ROM), an erasable programmable read only memory (EPROM or flash memory), an optical fiber, a portable compact disc read only memory (CD-ROM), an optical storage device, a magnetic storage device, or any suitable combination of the foregoing.

To provide interaction with a user, the systems and technologies described here may be implemented on a computer having: a display apparatus (for example, a cathode ray tube (CRT) or liquid crystal display (LCD) monitor) for displaying information to a user; and a keyboard and a pointing apparatus (for example, a mouse or a trackball) by which a user may provide input for the computer. Other kinds of apparatuses may also be used to provide interaction with a user; for example, feedback provided for a user may be any form of sensory feedback (for example, visual feedback, auditory feedback, or tactile feedback); and input from a user may be received in any form (including acoustic, speech or tactile input).

The systems and technologies described here may be implemented in a computing system (for example, as a data server) which includes a back-end component, or a computing system (for example, an application server) which includes a middleware component, or a computing system (for example, a user computer having a graphical user interface or a web browser through which a user may interact with an implementation of the systems and technologies described here) which includes a front-end component, or a computing system which includes any combination of such back-end, middleware, or front-end components. The components of the system may be interconnected through any form or medium of digital data communication (for example, a communication network). Examples of the communication network include: a local area network (LAN), a wide area network (WAN) and the Internet.

A computer system may include a client and a server. Generally, the client and the server are remote from each other and interact through the communication network. The relationship between the client and the server is generated by virtue of computer programs which run on respective computers and have a client-server relationship to each other.

It should be understood that various forms of the flows shown above may be used and reordered, and steps may be added or deleted. For example, the steps described in the present application may be executed in parallel, sequentially, or in different orders, which is not limited herein as long as the desired results of the technical solution disclosed in the present disclosure may be achieved.

The above-mentioned implementations are not intended to limit the scope of the present disclosure. It should be understood by those skilled in the art that various modifications, combinations, sub-combinations and substitutions may be made, depending on design requirements and other factors. Any modification, equivalent substitution and improvement made within the spirit and principle of the present disclosure all should be included in the extent of protection of the present disclosure.

1. A method for establishing a risk prediction model, comprising:
acquiring training data, the training data comprising a sample region set and annotation results of a risk grade of each sample region in the sample region set and a risk grade of a district to which each sample region belongs; and
training an initial model comprising an encoder, a discriminator and a classifier using the training data, and obtaining the risk prediction model using the encoder and the classifier in the initial model after the training process;
wherein the encoder performs a coding operation using region features of the sample regions to obtain a feature representation of each sample region; the discriminator identifies the risk grade of the district to which the sample region belongs according to the feature representation of the sample region; the classifier identifies the risk grade of the sample region according to the feature representation of the sample region; the initial model has training targets of minimizing a difference of identification of the sample regions belonging to the districts with different risk grades by the discriminator, and minimizing a difference between the identification result of the sample region by the classifier and the annotation result.

2. The method according to claim 1, wherein the region feature of the sample region comprises at least one of:
a surrounding preset-type POI feature, a demographic feature, and a user travel feature.

3. The method according to claim 1, wherein the surrounding preset-type POI feature comprises at least one of: information of a distance between the sample region and a nearest POI of a preset type, and a completeness degree of living facilities in a preset distance range of the sample region;
the demographic feature comprises at least one of: a population density condition, a commuting distance distribution, an age distribution, a gender distribution, an income distribution, a consumption ability distribution, an education level distribution, a marital status distribution, a life stage distribution, a job type distribution and an industry type distribution;
the user travel feature comprises at least one of: a travel mode, a starting point-destination mode distribution, and a starting point-travel mode-destination mode distribution.

4. The method according to claim 1, wherein the initial model further comprises a decoder;
the decoder reconstructs the region feature according to the feature representation of the sample region;
the training process also has a target of minimizing a difference between the region feature reconstructed by the decoder and the region feature extracted from the sample region.

5. The method according to claim 4, wherein in the process of training the initial model, parameters of the discriminator are optimized using a first loss function, parameters of the encoder are optimized using a second loss function, a third loss function and a fourth loss function, parameters of the classifier are optimized using the third loss function, and parameters of the decoder are optimized using the fourth loss function;
the first loss function is used to minimize a difference between a result of identification of the sample region by the discriminator and the annotation result;
the second loss function is used to minimize the difference of the identification of the sample regions belonging to the districts with different risk grades by the discriminator;
the third loss function is used to minimize the difference between the result of the identification of the sample region by the classifier and the annotation result;
the fourth loss function is used to minimize the difference between the region feature reconstructed by the decoder and the region feature extracted from the sample region.

6. A regional risk prediction method, comprising:
extracting region features of a target block; and
inputting the region features into a risk prediction model, and determining a risk grade of the target region according to a result output by the risk prediction model;
wherein the risk prediction model is pre-established by:
acquiring training data, the training data comprising a sample region set and annotation results of a risk grade of each sample region in the sample region set and a risk grade of a district to which each sample region belongs; and
training an initial model comprising an encoder, a discriminator and a classifier using the training data, and obtaining the risk prediction model using the encoder and the classifier in the initial model after the training process;
wherein the encoder performs a coding operation using region features of the sample regions to obtain a feature representation of each sample region; the discriminator identifies the risk grade of the district to which the sample region belongs according to the feature representation of the sample region; the classifier identifies the risk grade of the sample region according to the feature representation of the sample region; the initial model has training targets of minimizing a difference of identification of the sample regions belonging to the districts with different risk grades by the discriminator, and minimizing a difference between the identification result of the sample region by the classifier and the annotation result.

7. The method according to claim 6, wherein the risk grade is a risk grade of epidemic spread.

8-14. (canceled)

15. An electronic device, comprising:
at least one processor; and
a memory connected with the at least one processor communicatively;
wherein the memory stores instructions executable by the at least one processor to cause the at least one processor to perform a method for establishing a risk prediction model, which comprises:
acquiring training data, the training data comprising a sample region set and annotation results of a risk grade of each sample region in the sample region set and a risk grade of a district to which each sample region belongs; and
training an initial model comprising an encoder, a discriminator and a classifier using the training data, and obtaining the risk prediction model using the encoder and the classifier in the initial model after the training process;
wherein the encoder performs a coding operation using region features of the sample regions to obtain a feature representation of each sample region; the discriminator identifies the risk grade of the district to which the sample region belongs according to the feature representation of the sample region; the classifier identifies the risk grade of the sample region according to the feature representation of the sample region; the initial model has training targets of minimizing a difference of identification of the sample regions belonging to the districts with different risk grades by the discriminator, and minimizing a difference between the identification result of the sample region by the classifier and the annotation result.

16. A non-transitory computer readable storage medium comprising computer instructions, which, when executed by a computer, cause the computer to perform a method for establishing a risk prediction model, which comprises:
acquiring training data, the training data comprising a sample region set and annotation results of a risk grade of each sample region in the sample region set and a risk grade of a district to which each sample region belongs; and
training an initial model comprising an encoder, a discriminator and a classifier using the training data, and obtaining the risk prediction model using the encoder and the classifier in the initial model after the training process;
wherein the encoder performs a coding operation using region features of the sample regions to obtain a feature representation of each sample region; the discriminator identifies the risk grade of the district to which the sample region belongs according to the feature representation of the sample region; the classifier identifies the risk grade of the sample region according to the feature representation of the sample region; the initial model has training targets of minimizing a difference of identification of the sample regions belonging to the districts with different risk grades by the discriminator, and minimizing a difference between the identification result of the sample region by the classifier and the annotation result.

17. (canceled)

18. The electronic device according to claim 15, wherein the region feature of the sample region comprises at least one of:
a surrounding preset-type POI feature, a demographic feature, and a user travel feature.

19. The electronic device according to claim 15, wherein the surrounding preset-type POI feature comprises at least one of: information of a distance between the sample region and a nearest POI of a preset type, and a completeness degree of living facilities in a preset distance range of the sample region;
the demographic feature comprises at least one of: a population density condition, a commuting distance distribution, an age distribution, a gender distribution, an income distribution, a consumption ability distribution, an education level distribution, a marital status distribution, a life stage distribution, a job type distribution and an industry type distribution;
the user travel feature comprises at least one of: a travel mode, a starting point-destination mode distribution, and a starting point-travel mode-destination mode distribution.

20. The electronic device according to claim 15, wherein the initial model further comprises a decoder;
the decoder reconstructs the region feature according to the feature representation of the sample region;
the training process also has a target of minimizing a difference between the region feature reconstructed by the decoder and the region feature extracted from the sample region.

21. The electronic device according to claim 20, wherein in the process of training the initial model, parameters of the discriminator are optimized using a first loss function, parameters of the encoder are optimized using a second loss function, a third loss function and a fourth loss function, parameters of the classifier are optimized using the third loss function, and parameters of the decoder are optimized using the fourth loss function;
the first loss function is used to minimize a difference between a result of identification of the sample region by the discriminator and the annotation result;
the second loss function is used to minimize the difference of the identification of the sample regions belonging to the districts with different risk grades by the discriminator;
the third loss function is used to minimize the difference between the result of the identification of the sample region by the classifier and the annotation result;
the fourth loss function is used to minimize the difference between the region feature reconstructed by the decoder and the region feature extracted from the sample region.

22. The non-transitory computer readable storage medium according to claim 16, wherein the region feature of the sample region comprises at least one of:
a surrounding preset-type POI feature, a demographic feature, and a user travel feature.

23. The non-transitory computer readable storage medium according to claim 16, wherein the surrounding preset-type POI feature comprises at least one of: information of a distance between the sample region and a nearest POI of a preset type, and a completeness degree of living facilities in a preset distance range of the sample region;
the demographic feature comprises at least one of: a population density condition, a commuting distance distribution, an age distribution, a gender distribution, an income distribution, a consumption ability distribution, an education level distribution, a marital status distribution, a life stage distribution, a job type distribution and an industry type distribution;
the user travel feature comprises at least one of: a travel mode, a starting point-destination mode distribution, and a starting point-travel mode-destination mode distribution.

24. The non-transitory computer readable storage medium according to claim 16, wherein the initial model further comprises a decoder;
the decoder reconstructs the region feature according to the feature representation of the sample region;
the training process also has a target of minimizing a difference between the region feature reconstructed by the decoder and the region feature extracted from the sample region.

25. The non-transitory computer readable storage medium according to claim 24, wherein in the process of training the initial model, parameters of the discriminator are optimized using a first loss function, parameters of the encoder are optimized using a second loss function, a third loss function and a fourth loss function, parameters of the classifier are optimized using the third loss function, and parameters of the decoder are optimized using the fourth loss function;
the first loss function is used to minimize a difference between a result of identification of the sample region by the discriminator and the annotation result;
the second loss function is used to minimize the difference of the identification of the sample regions belonging to the districts with different risk grades by the discriminator;
the third loss function is used to minimize the difference between the result of the identification of the sample region by the classifier and the annotation result;
the fourth loss function is used to minimize the difference between the region feature reconstructed by the decoder and the region feature extracted from the sample region.
